SEQUENCE LISTING (21 sequences)

SEQ ID NO: 1 - protein, 10 aa, Artificial Sequence
  1Ab VH CDR1
GFTFSSYTMS

SEQ ID NO: 2 - protein, 17 aa, Artificial Sequence
  1Ab VH CDR2
YISNGGGSISYPDTVKG

SEQ ID NO: 3 - protein, 16 aa, Artificial Sequence
  1Ab VH CDR3
EGLRPGDRDRYYAMDY

SEQ ID NO: 4 - protein, 125 aa, Artificial Sequence
  1Ab VH
EVQLQESGGGLVQPGGSLKLSCAASGFTFSSYTMSWVRQTPEKRLEWVAYISNGGGSISYPDTVKGRFTISRDNAKNTLYLHMSSLKSEDTAMYHCAREGLRPGDRDRYYAMDYWGQGTSVTVSS

SEQ ID NO: 5 - protein, 12 aa, Artificial Sequence
  1Ab VL CDR1
SVSSSVSSSNLH

SEQ ID NO: 6 - protein, 7 aa, Artificial Sequence
  1Ab VL CDR2
GTSTLAS

SEQ ID NO: 7 - protein, 9 aa, Artificial Sequence
  1Ab VL CDR3
QQWSSYPLT

SEQ ID NO: 8 - protein, 109 aa, Artificial Sequence
  1Ab VL
DIVITQSPALMAAFPGDRVTITCSVSSSVSSSNLHWYRQKSETSPKPWIYGTSTLASGVPVRFSGSGSGTSYSLTISSMEAEDAATYYCQQWSSYPLTFGAGTKLELKR

SEQ ID NO: 9 - protein, 10 aa, Artificial Sequence
  2Ab VH CDR 1
GFTFSTYYMS

SEQ ID NO: 10 - protein, 17 aa, Artificial Sequence
  2Ab VH CDR2
AINSNGGGTYYPDNVKG

SEQ ID NO: 11 - protein, 10 aa, Artificial Sequence
  2Ab VH CDR3
YFGSTYAMDY

SEQ ID NO: 12 - protein, 119 aa, Artificial Sequence
  2Ab VH
EVQLQESGGGLVKLGGSLKLSCAASGFTFSTYYMSWVRQTPEKRLELVAAINSNGGGTYYPDNVKGRFTISRDNAKNTLYLQMSSLKSEDTALYYCTNYFGSTYAMDYWGQGTSVTVSS

SEQ ID NO: 13 - protein, 16 aa, Artificial Sequence
  2Ab VL CDR1
RSSQSLVHSNGNTYLH

SEQ ID NO: 14 - protein, 7 aa, Artificial Sequence
  2Ab VL CDR2
KVSNRFS

SEQ ID NO: 15 - protein, 9 aa, Artificial Sequence
  2Ab VL CDR3
SQNTHVPFT

SEQ ID NO: 16 - protein, 113 aa, Artificial Sequence
  2Ab VL
DIVLTQSPLSLPVSLGDQASISCRSSQSLVHSNGNTYLHWYLQKPGQSPKLLIYKVSNRFSGVPDRFSGSGLGTDFTLKIIRVEAEDLGVYFCSQNTHVPFTFGSGTKLEIKR

SEQ ID NO: 17 - DNA, 375 bp, Artificial Sequence
  1Ab VH DNA
gaggtgcaactgcaggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagtatcagctatccagacactgtaaagggccgattcaccatctccagagacaatgccaagaacaccctgtatctgcacatgagcagtctgaagtctgaggacacggccatgtatcattgtgcaagagaggggttacgacccggggaccgcgataggtactatgctatggactactggggtcaaggaacctcagtcaccgtctcctca

SEQ ID NO: 18 - DNA, 327 bp, Artificial Sequence
  1Ab VL DNA
gatattgtgataacccagtctccagcactcatggctgcatttccaggggacagggtcaccatcacctgcagtgtcagctcaagtgtaagttccagcaacttgcactggtaccgacagaagtcagaaacctcccccaaaccctggatttatggcacatccaccctggcttctggagtccctgttcgcttcagtggcagtggatctgggacctcttactctctcacaatcagcagcatggaggctgaagatgctgccacttattactgtcaacagtggagtagttacccactcacgttcggtgctgggaccaagctggagctgaaacgg

SEQ ID NO: 19 - DNA, 357 bp, Artificial Sequence
  2Ab VH DNA
gaggtgcagctgcaggagtctgggggaggcttagtgaagcttggagggtccctgaaactctcctgtgcagcctctggattcactttcagtacttattacatgtcttgggttcgccagactccagagaagaggctggagttggtcgcagccattaatagtaatggtggtggcacctactatccagacaatgtgaagggccgattcaccatctccagagacaatgccaagaacaccctgtacctgcaaatgagcagtctgaagtctgaggacacagccttgtattactgtacaaattacttcggtagtacctacgctatggactactggggtcaaggaacctcagtcaccgtctcctca

SEQ ID NO: 20 - DNA, 339 bp, Artificial Sequence
  2Ab VL DNA
gacattgtgctgacccaatctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggattagggacagatttcacactcaagatcatcagagtggaggctgaggatctgggagtttatttctgctctcaaaatacacatgttccattcacgttcggctcggggacaaagttggaaataaagcgg

SEQ ID NO: 21 - protein, 988 aa, Artificial Sequence
  268th to 1255th amino acid residues of SARS CoV spike protein
ENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

(19) United States
(12) Patent Application Publication
(10) Pub. No.: US 2022/0089691 A1
(43) Pub. Date: Mar. 24, 2022

(54) ANTI-SARS-COV-2 ANTIBODIES AND APPLICATION THEREOF

(71) Applicant: National Defense Medical Center, Taipei (TW)

(72) Inventors: Szu-Chia LAI, New Taipei (TW); Yu-Yine Huang, New Taipei (TW); Yi-Ling Chen, Taoyuan (TW); Jiunn-Jye Wey, New Taipei (TW); Po-Shiuan Hsieh, Taipei (TW); Meng-Hung Tsai, Taipei (TW); Jyh-Hwa Kau, Taoyuan (TW)

(21) Appl. No.: 17/021,042

(22) Filed: Sep. 15, 2020

(51) Int. Cl. C07K 16/10; G01N 33/563

(52) U.S. Cl. CPC C07K 16/10; C07K 2317/565; G01N 33/563

(57) ABSTRACT

The present invention relates to anti-SARS-CoV-2 antibodies and/or antigen-binding fragments thereof, which are useful for specific detection of SARS-CoV-2 in a biological sample. The present invention also provides methods and kits for detecting SARS-CoV-2 and methods and compositions for use in diagnosis and treatment of coronavirus disease (COVID-19) using the anti-SARS-CoV-2 antibodies as described herein.

### BACKGROUND OF THE INVENTION

### 1. Field of the Invention

The present invention relates to anti-SARS-CoV-2 antibodies and/or antigen-binding fragments thereof, which are useful for specific detection of SARS-CoV-2 in a biological sample. The present invention also provides methods and kits for detecting SARS-CoV-2 and methods and compositions for use in diagnosis and treatment of coronavirus disease (COVID-19) using the anti-SARS-CoV-2 antibodies as described herein.

### 2. Description of the Prior Art

On 31 Dec. 2019, the World Health Organization (WHO) was alerted to several cases of pneumonia in Wuhan City, Hubei Province of China. The viral pathogen did not match any other known virus and was later officially named “severe acute respiratory syndrome coronavirus 2 (SARS-CoV-2).” The official name of the disease caused by SARS-CoV-2 is coronavirus disease 2019 (COVID-19). Common symptoms of COVID-19 include fever, dry cough, fatigue, tiredness, muscle or body aches, sore throat, diarrhea, conjunctivitis, headache, loss of taste or smell, a rash on skin, and shortness of breath. While the majority of cases result in mild symptoms, some progress to acute respiratory distress syndrome (ARDS), precipitated by cytokine storm, multi-organ failure, septic shock, and blood clots.

Although the first known human infections of SARS-CoV-2 were in China, the COVID-19 pandemic has been impacting numerous people worldwide socially and financially. As of 23 August 2020, over 23 million confirmed cases of COVID-19, including more than 800 thousand deaths, have been reported to the WHO. The numbers are still growing fast.

As so far, no effective antiviral agents are currently available to treat COVID-19 and vaccine still in the phase of evaluation. Therefore, the diagnosis of COVID-19 in early stage is very important for disease management. The early diagnosis of SARS-CoV-2 infection would provide intervention to treat patients and control the epidemics. People infected with SARS-CoV-2 exhibit a wide range of clinical presentations, ranging from asymptomatic infections to severe pneumonia, making accurate diagnoses difficult. Current diagnostic tests for SARS-CoV-2 are limited in laboratories, including isolation and culture of the virus, viral RNA detection, antigen detection, and antibody detection. Isolation and culture of SARS-CoV-2 must be performed in a biosafety level 3 (BSL-3) laboratory, which makes the method difficult and time-consuming. Antigen and antibody detections are still under development, so the major diagnosis of SARS-CoV-2 is still based on the detection of viral nucleic acid. However, nucleic acid detection requires professional operators, special instruments, and equipment, and it takes more than 4 hours to finish the test. Therefore, there is an urgent need to develop a diagnostic method that can quickly detect SARS CoV-2.

### SUMMARY OF THE INVENTION

The present invention is based on the identification of a number of isolated antibodies, which unexpectedly exhibit superior specificity for Severe Acute Respiratory Syndrome coronavirus (SARS-CoV) and SARS-CoV-2. Each of the antibody of the present invention is capable of detecting SARS-CoV and SARS-CoV-2 without substantially cross reacting with other respiratory viruses. The present invention provides such antibodies and antigen-binding fragment thereof and also compositions and kits comprising the same and methods using the same. The present invention is useful for specific detection of SARS-CoV and/or SARS-CoV-2 in a sample, particularly a sample from a patient suspected to be infected by the virus.

In one aspect, the present invention provides an isolated antibody against SARS-CoV-2 or antigen-binding fragment thereof, wherein the isolated antibody is selected from the group consisting of:
(i) a first antibody, comprising
   (a) a heavy chain variable region (V_{H}) which comprises a heavy chain complementarity determining region 1 (HC CDR1) of SEQ ID NO: 1, a heavy chain complementarity determining region 2 (HC CDR2) of SEQ ID NO: 2, and a heavy chain complementarity determining region 3 (HC CDR3) of SEQ ID NO: 3; and
   (b) a light chain variable region (V_{L}) which comprises a light chain complementarity determining region 1 (LC CDR1) of SEQ ID NO: 5, a light chain complementarity determining region 2 (LC CDR2) of SEQ ID NO: 6, and a light chain complementarity determining region 3 (LC CDR3) of SEQ ID NO: 7; and
(ii) a second antibody, comprising
   (a) a V_{H} which comprises an HC CDR1 of SEQ ID NO: 9, an HC CDR2 of SEQ ID NO: 10, and an HC CDR3 of SEQ ID NO: 11; and
   (b) a V_{L} which comprises an LC CDR1 of SEQ ID NO: 13, an LC CDR2 of SEQ ID NO: 14, and an LC CDR3 of SEQ ID NO: 15; and
(iii) a combination of (i) and (ii).

In some embodiments, the first antibody comprises a V_{H} comprising SEQ ID NO: 4 and a V_{L} comprising SEQ ID NO: 8.

In some embodiments, the second antibody comprises a V_{H} comprising SEQ ID NO: 12 and a V_{L} comprising SEQ ID NO: 16.

The SARS-CoV-2 specific antibodies of the present invention can be a full-length antibody. The antigen-binding fragment of the antibodies of the present invention can be scFv, (scFv)₂, Fab, Fab′, F(ab′)₂.

In another aspect, the present invention provides a nucleic acid comprising a nucleotide sequence encoding an antibody heavy chain variable region (V_{H}) or an antibody light chain variable region (V_{L}) or both, wherein the V_{H} and V_{L} are as described herein.

The present invention also provides a vector (e.g. an expression vector) comprising any of the nucleic acids described herein and a host cell comprising such a vector.

The present invention further provides a method for preparing an antibody specific to SARS-CoV-2, comprising (i) culturing the host cell as described herein under conditions allowing for expression of the antibody, and optionally (ii) harvesting the antibody from the cell culture.

In a further aspect, the present invention provides a composition comprising (a) any of the antibody specific to SARS-CoV-2 as described herein, any of the nucleic acids as described herein, or any of the vectors as described herein; and (b) a carrier, such as a pharmaceutically acceptable carrier.

In some embodiments, the composition of the present invention is a pharmaceutical composition for use in treatment of COVID-19.

In some embodiments, the composition of the present invention is a diagnostic composition for use in diagnosis of COVID-19.

In still another aspect, the present invention provides a method for detecting SARS-CoV-2 in a sample suspected of containing said SARS-CoV-2, comprising contacting the sample with an isolated antibody or antigen-binding fragment thereof specific to SARS-CoV-2, as described herein, and assaying binding of the antibody with the sample. The binding results are to determine the presence of respective SARS-CoV-2 serotypes in the sample. In some embodiments, the antibodies of the present invention are paired with each other to perform a sandwich assay.

In further another aspect, the present invention provides a kit for detecting the presence of SARS-CoV-2 in a sample, comprising one or more antibodies specific to SARS-CoV-2 as described herein.

In some embodiments, at least one of the antibodies in the kit comprises a detectable label.

Examples of the detectable label include, but are not limited to, an enzymatic label, a fluorescent label, a metal label and a radio label.

In some embodiments, the kit is an immunoassay kit.

Examples of the immunoassay include, but are not limited to, ELISA (enzyme-linked immunosorbent assay), RIA (radioimmunoassay), FIA (fluorescence immunoassay), LIA (luminescence immunoassay), or ILMA (immunoluminometric assay).

In certain embodiments, the immunoassay is in a lateral flow assay format.

In particular, the immunoassay is a sandwich assay.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A shows the result (400x) of indirect immunofluorescence assay (IFA) using monoclonal antibody (mAb) 8a-1 of the present invention to detect Vero E6 cells infected with SARS-CoV-2. FIG. 1B shows the result (400x) of IFA using monoclonal antibody mAb27b-2 of the present invention to detect Vero E6 cells infected with SARS-CoV-2. FIG. 1C shows the result (400x) of IFA using serum from a patient infected with SARS-CoV-2 to detect Vero E6 cells infected with SARS-CoV-2. FIG. 1D shows the result (400x) of IFA using anti-dengue virus monoclonal antibody, anti-D2 NS1, to detect Vero E6 cells infected with SARS-CoV-2.

FIG. 2 shows the results of detection in the sandwich (capture) ELISA using mAb8a-1 of the present invention as the capture antibodies, paired with mAb27b-2 of the present invention as the detection antibodies. In this assay, cell lysate from Vero E6 cells infected with SARS-CoV or SARS-CoV-2, and viral culture supernatants from human coronavirus (HCoV)-229E, HCoV-OC43, influenza A (Flu A) H1N1, Flu A H3N2, influenza B (Flu B), Adenovirus-7a, Parainfluenza-3, Rhinovirus, Respiratory Syncytial Virus (RSV)-A2 were used as sources of spike protein, and supernatant from non-infected Vero E6 cells was used as a negative control.

FIG. 3 shows the sensitivities of the sandwich (capture) ELISA using mAb8a-1 of the present invention as the capture antibodies, paired with mAb27b-2 of the present invention as the detection antibodies. Capture ELISA was performed with serial diluted and purified S2 protein to determine the detection limits of the mAbs of the present invention. Bovine serum albumin (BSA) was used to establish the baseline. Each data point represents the mean±Standard Deviation (SD) of three replicate tests.

FIG. 4A shows the basic design of the sandwich strip using mAb8a-1 of the present invention as the capture antibodies, paired with mAb27b-2 of the present invention as the detection antibodies.

FIG. 4B shows the basic design of the sandwich strip using mAb27b-2 of the present invention as the capture antibodies, paired with mAb8a-1 of the present invention as the detection antibodies.

FIG. 5 shows the specificities of the sandwich strip of the present invention. Monoclonal antibody (mAb) 8a-1 paired with mAb27b-2 were used as detection and capture antibodies, respectively, and then tested for binding specificity. In this assay, viral culture supernatants from SARS-CoV-2 (strain CoV19/34005-MK2/20200227), SARS-CoV-2 (strain CoV19/73611-MK2/20200314), HCoV-229E, HCoV-OC43, influenza A (strain A/CA/07/2009), influenza B (strain B/T/81863/2014), Parainfluenza, Adenovirus-7a, Rhinovirus, RSV were used as sources of spike protein, and supernatant from non-infected Vero E6 cells was used as a negative control. Arrows indicate positive reactions.

FIG. 6 shows the sensitivity of the sandwich strip of the present invention. Monoclonal antibody (mAb) 8a-1 paired with mAb27b-2 were used as detection and capture antibodies, respectively, and then tested for binding sensitivity. In this assay, spike protein at different concentrations (0, 62.5, 125, 250 ng/mL) was used as antigen. Arrows indicate positive reactions.

### DETAILED DESCRIPTION OF THE PREFERRED EMBODIMENT

The present invention relates to anti-SARS-CoV-2 antibodies. The present invention provides such antibodies and antigen-binding fragments thereof, which are useful for detection of SARS-CoV-2 in a biological sample. The present invention further provides methods and vectors for preparing the antibodies or antigen-binding fragments thereof, and also kits and compositions comprising the same and methods using the same for detection of SARS-CoV-2 and diagnosis and treatment of COVID-19.

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

I. Definitions

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, or 20 amino acid residues).

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form) means an immunoglobulin molecule having the ability to specifically bind to a particular target antigen. As used herein, the term “antibody” includes not only intact (i.e. full-length) antibody molecules but also antigen-binding fragments thereof retaining antigen binding ability e.g. Fab, Fab′, F(ab′)₂ and Fv. Such fragments are also well known in the art and are regularly employed both in vitro and in vivo. The term “antibody” also includes humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

An intact or complete antibody comprises two heavy chains and two light chains. Each heavy chain contains a variable region (V_{H}) and a first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables region in both chains generally contain three highly variable regions, called the complementarity determining regions (CDRs); namely, light (L) chain CDRs including LC CDR1, LC CDR2, and LC CDR3, and heavy (H) chain CDRs including HC CDR1, HC CDR2, HC CDR3). The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha (α), delta (δ), epsilon (ε), gamma (γ), and mu (μ), respectively.

As used herein, the term “antigen-binding domain” or “antigen-binding fragment” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab')2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain through a peptide linker; and (v) a (scFv)₂, which can comprise two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in these chimeric antibodies, the variable region of both light and heavy chains may mimic the variable region of antibodies derived from one species of mammal (e.g., a non-human mammal such as mouse, rabbit and rat), while the constant portions may be homologous to the sequences in antibodies derived from another mammal such as a human.

As used herein, the term “humanized antibody” refers to an antibody comprising a framework region from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. The human antibodies may include one or more amino acid residues not encoded by human germline immunoglobulin sequences e.g. by mutations in one or more of the CDRs, or in one or more of the FRs, so as to, for example, decrease possible immunogenicity, increase affinity, eliminate cysteines that might cause undesirable folding, etc.

As used herein, an “isolated” substance means that it has been altered by the hand of man from the natural state. In some embodiments, the polypeptide (e.g. antibody) or nucleic acids of the present invention can be said to be “isolated” or “purified” if they are substantially free of cellular material or chemical precursors or other chemicals/components that may be involved in the process of peptides/nucleic acids preparation. It is understood that the term “isolated” or “purified” does not necessarily reflect the extent to which the peptide has been “absolutely” isolated or purified e.g. by removing all other substances (e.g., impurities or cellular components). In some cases, for example, an isolated or purified polypeptide includes a preparation containing the polypeptide having less than 50%, 40%, 30%, 20% or 10% (by weight) of other proteins (e.g. cellular proteins), having less than 50%, 40%, 30%, 20% or 10% (by volume) of culture medium, or having less than 50%, 40%, 30%, 20% or 10% (by weight) of chemical precursors or other chemicals/components involved in synthesis procedures.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen or an epitope than it does with other targets/epitopes. An antibody “specifically binds” to a target antigen if it binds with greater affinity, avidity, more readily, and/or with greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means specific/preferential binding. The affinity of the binding is defined in terms of a dissociation constant (K_{D}). Typically, specifically binding when used with respect to an antibody can refer to an antibody that specifically binds to (recognize) its target with an K_{D} value less than about 10^{˜7} M, such as about 10^{˜8} M or less, such as about 10^{˜9} M or less, about 10^{˜10} M or less, about 10^{˜11} M or less, about 10^{˜12} M or less, or even less, and binds to the specific target with an affinity corresponding to a K_{D} that is at least ten-fold lower than its affinity for binding to a non-specific antigen (such as BSA or casein), such as at least 100 fold lower, for instance at least 1,000 fold lower, such as at least 10,000 fold lower.

As used herein, the term “cross-reactive” can refer to the ability of an antibody to react with similar antigenic sites on different proteins.

As used herein, the term “Coronavirus” refers to viruses belonging to the family Coronavirinae. Coronaviruses are enveloped RNA viruses that are spherical in shape and characterized by crown-like spikes on the surface under an electron microscope, hence the name. This type of virus can be further divided into four subgroups: alpha (α), beta (β), gamma (γ), and delta (δ). There are seven human coronavirus strains, including two alpha coronaviruses (HCov-229E and HCoV-NL63), two beta coronaviruses (HCov-HKU1 and HCov-OC43), Middle East respiratory syndrome coronavirus (MERS-CoV), SARS-CoV, and the newly discovered SARS-CoV-2.

As used herein, the term “Severe acute respiratory syndrome coronavirus 2 (SARS-CoV-2)” refers to the strain of coronavirus that causes Coronavirus disease 2019 (COVID-19). SARS-CoV-2 is a positive-sense single-stranded RNA virus that is a member of the genus Betacoronavirus of the family Coronavirinae. The RNA sequence of SARS-CoV-2 is approximately 30,000 bases in length. Each SARS-CoV-2 virion is 50-200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope.

As used herein, the term “Coronavirus disease 2019,” “COVID-19,” “2019-nCoV acute respiratory disease,” “Novel coronavirus pneumonia,” or “Severe pneumonia with novel pathogens,” which can be used interchangeably, refers to the disease caused by SARS CoV-2. The virus primarily spreads between people through close contact and via respiratory droplets produced from coughs or sneezes. People with COVID-19 may have no symptom or a wide range of symptoms, ranging from mild symptoms to severe illness. Symptoms may appear 1-14 days after exposure to SARS CoV-2. Most common symptoms of COVID-19 include fever, dry cough, and tiredness, less common symptoms include aches and pains, sore throat, diarrhea, conjunctivitis, headache, loss of taste or smell, and a rash on skin, or discolouration of fingers or toes, and serious symptoms include difficulty breathing or shortness of breath, chest pain or pressure, and loss of speech or movement. While most people have mild symptoms, some people develop acute respiratory distress syndrome (ARDS), multi-organ failure, septic shock, and even death.

As used herein, the term “spike protein,” “S polypeptide,” or “S protein,” which can be used interchangeably, refers to a surface structure glycoprotein on SARS CoV-2 and is responsible for allowing the virus to attach to and fuse with the membrane of a host cell. Each monomer of trimeric S protein is about 180 kDa, and contains two subunits, S1 and S2, mediating attachment and membrane fusion, respectively. Spike protein mainly enters human cells by binding to the receptor angiotensin converting enzyme 2 (ACE2). Amino acid sequences of SARS CoV-2 Spike protein are known and available in the art, such as, but are not limited to, GenBank accession number QJH92179.1, QHD43416.1, QJH92167.1, and QKU37045.1.

The term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-TATAG-3′ is complementary to a polynucleotide whose sequence is 5′-CTATA-3′.”

The term “encoding” refers to the inherent property of specific sequences of nucleotides in a polynucleotide (e.g., a gene, a cDNA, or an mRNA) to serve as templates for synthesis of other polymers and macromolecules in biological processes having either a given sequence of nucleotides (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

The term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, fosmids, episomes, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

The term “individual” or “subject” used herein includes human and non-human animals such as companion animals (such as dogs, cats and the like), farm animals (such as cows, sheep, pigs, horses and the like), or laboratory animals (such as rats, mice, guinea pigs and the like).

The term “treating” as used herein refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder or the symptom or condition thereof.

The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

II. Antibodies Specific to SARS-CoV-2

The present invention is based on the identification of a number of isolated antibodies specific to SARS-CoV-2, including mAb8a-1 and mAb27b-2. These anti-SARS-CoV-2 antibodies were found to be capable of recognizing SARS-CoV and SARS-CoV-2 without cross reacting with other respiratory viruses. More specifically, these anti-SARS-CoV-2 antibodies are specific to SARS-CoV-2 Spike protein. These anti-SARS-CoV-2 antibodies were found effective and workable in various forms of immunoassays to detect SARS-CoV-2 in a sample with superior sensitivity and specificity.

Accordingly, described herein are anti-SARS-CoV-2 antibodies, including mAb8a-1 and mAb27b-2, and functional variants thereof. The reactivity of these mAbs with SARS-CoV-2 was determined using ELISA and Western blot. The characterizations of these mAbs are showed in Table 1. The amino acid sequences of the heavy chain variable region (V_{H}) and light chain variable region (V_{L}) of each of mAb8a-1 and mAb27b-2 are as shown in SEQ ID NOs: 4, 8, 12, and 16, respectively.

A functional variant of mAb8a-1 (also referred to “a first antibody” as described herein) can comprise

(a) a heavy chain variable region (V_{H}) which comprises a heavy chain complementarity determining region 1 (HC CDR1) of SEQ ID NO: 1, a heavy chain complementarity determining region 2 (HC CDR2) of SEQ ID NO: 2, and a heavy chain complementarity determining region 3 (HC CDR3) of SEQ ID NO: 3; and

(b) a light chain variable region (V_{L}) which comprises a light chain complementarity determining region 1 (LC CDR1) of SEQ ID NO: 5, a light chain complementarity determining region 2 (LC CDR2) of SEQ ID NO: 6, and a light chain complementarity determining region 3 (LC CDR3) of SEQ ID NO: 7.

In some embodiments, the first antibody comprises a V_{H} comprising SEQ ID NO: 4 or an amino acid sequence substantially identical thereto and a V_{L} comprising SEQ ID NO: 8 or an amino acid sequence substantially identical thereto. Specifically, the first antibody includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO: 4, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO: 8. The first antibody also includes any recombinantly (engineered)-derived antibody encoded by the polynucleotide sequence encoding the relevant V_{H} or V_{L} amino acid sequences as described herein.

A functional variant of mAb27b-2 (also referred to “a second antibody” as described herein) can comprise
(a) a V_{H} which comprises an HC CDR1 of SEQ ID NO: 9, an HC CDR2 of SEQ ID NO: 10, and an HC CDR3 of SEQ ID NO: 11; and
(b) a V_{L} which comprises an LC CDR1 of SEQ ID NO: 13, an LC CDR2 of SEQ ID NO: 14, and an LC CDR3 of SEQ ID NO: 15; and
(iii) a combination of (i) and (ii).

In some embodiments, the second antibody comprises a V_{H} comprising SEQ ID NO: 12 or an amino acid sequence substantially identical thereto and a V_{L} comprising SEQ ID NO: 16 or an amino acid sequence substantially identical thereto. Specifically, the second antibody includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO: 12, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO: 16. The second antibody also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may be monoclonal or polyclonal. A “monoclonal antibody” refers to a homogenous antibody population, and a “polyclonal antibody” refers to a heterogeneous antibody population. The antibodies described herein may also include their antigen-binding fragments e.g. a Fab fragment, a F(ab′)2 fragment, a Fv fragment, a single chain Fv (scFv) and a (scFv)₂. The antibodies or their antigen-binding fragments can be prepared by methods known in the art.

III. Preparation of Antibodies

Numerous methods conventional in this art are available for obtaining antibodies or antigen-binding fragments thereof.

In some embodiments, the antibodies provided herein may be made by the conventional hybridoma technology. In general, a target antigen, e.g. a SARS-CoV-2 spike protein, optionally coupled to a carrier protein, e.g. keyhole limpet hemocyanin (KLH), and/or mixed with an adjuvant, e.g complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of mAb8a-1 and mAb27b-2 as described herein are as shown in SEQ ID NOs: 17, 18, 19, and 20, respectively. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

IV. Use of Antibodies

The anti-SARS-CoV-2 antibodies of the present invention are specific to SARS-CoV and SARS-CoV-2, and each of which does not react with any other respiratory viruses, such as human coronavirus (HCoV), influenza A (Flu A), influenza B (Flu B), Adenovirus, Parainfluenza, Rhinovirus, and Respiratory Syncytial Virus (RSV). The present invention thus provides a method employing any of the disclosed antibodies or any combination thereof that can be effectively employed to detect SARS-CoV-2 in a sample.

In general, the method of the present invention comprises contacting the sample with any of the disclosed antibodies or any combination thereof and assaying binding of the antibody with said sample. Particularly, the binding of the antibody with said sample includes (i) binding of a first antibody to SARS-CoV-2 spike protein and forming a first antibody-SARS-CoV-2 spike protein complex, and (ii) binding of a second antibody to SARS-CoV-2 spike protein and forming a second antibody-SARS-CoV-2 spike protein complex. The method of the present invention further comprises determining the presence of SARS-CoV-2 based on the binding of the antibody with the sample.

There are various assay formats known to those of ordinary skill in the art for using antibodies to detect an antigen or pathogen in a sample. These assays that use antibodies specific to target antigens/pathogens are generally called immunoassays. Examples of immunoassays include but are not limited to ELISA (enzyme-linked immunosorbent assay), RIA (radioimmunoassay), FIA (fluorescence immunoassay), LIA (luminescence immunoassay), or immunoluminometric assay (ILMA). Such assays can be employed to detect the presence of SARS-CoV-2 in biological samples including blood, serum, plasma, saliva, cerebrospinal fluid, urine, and other tissue specimens.

In certain embodiments, the assay is a sandwich assay.

In some embodiments, the assay is performed by first immobilizing a capture antibody on a solid support. The immobilized antibody is then incubated with the biological sample, and the SARS-CoV-2 or its target antigen e.g. spike protein (if present in the sample) is allowed to bind to the antibody, to form an antibody-virus/antigen complex or conjugate. Unbound sample can then be removed by washing the solid support with an appropriate buffer, such as phosphate-buffered saline (PBS) containing 0.05% Tween, and a detection antibody that can bind to the immobilized antibody-virus/antigen complex and comprises a detectable label is added to the solid support. Specifically, the capture antibody and the detection antibody need to bind to the virus/antigen at different epitopes such that the virus/antigen can be “sandwiched” between the two antibodies. Preferred detectable labels include an enzymatic label (such as horseradish peroxidase), a fluorescent label, a metal label and a radio label. Some particular examples of detectable labels include gold nanoparticles, colored latex beads, magnetic particles, carbon nanoparticles and selenium nanoparticles. The detection antibody is incubated with the immobilized antibody-virus/antigen complex for a period of time sufficient to detect the bound virus/antigen. Unbound detection antibody is then removed and bound detection antibody is detected based on the detectable label. For example, an enzymatic label may be generally be detected by the addition of substrate, followed by spectroscopic or other analysis of the reaction products.

To determine the presence of target SARS-CoV-2 in the sample, the signal detected from the detectable label bound to the solid support is generally compared to a signal that corresponds to a cut-off value. This cut-off value is typically the average mean signal obtained when the immobilized antibody is incubated with samples from an uninfected subject. In general, a sample generating a signal that is higher than the cut-off value is considered positive for the presence of SARS-CoV-2 in the sample.

An anti-SARS-CoV-2 antibody of the present invention can be used as capture or detection antibodies, paired with another anti-SARS-CoV-2 antibody of the present invention, to form an antibody pair to perform a sandwich assay. The other anti-SARS-CoV-2 antibody as described herein needs to be able to also bind to the target virus/antigen but at a different epitope than the anti-SARS-CoV-2 antibody of the present invention as used in the pair for performing a sandwich assay.

In some embodiments, the method of the present invention for detecting SARS-CoV-2 in a sample comprises
(i) contacting the sample with a first anti-SARS-CoV-2 antibody, as a capture antibody, to form a first antibody-SARS-CoV-2 spike protein complex;
(ii) contacting the first antibody-SARS-CoV-2 spike protein complex with a second anti-SARS-CoV-2 antibody, as a detection antibody, to form a first antibody-SARS-CoV-2 spike protein-second antibody complex; and
(iii) detecting the presence of the first antibody-SARS-CoV-2 spike protein-second antibody complex, thereby detecting the presence of SARS-CoV-2 in the sample.

In some embodiments, the method of the present invention for detecting SARS-CoV-2 in a sample comprises
(i) contacting the sample with a second anti-SARS-CoV-2 antibody, as a capture antibody, to form a second antibody-SARS-CoV-2 spike protein complex;
(ii) contacting the second antibody-SARS-CoV-2 spike protein complex with a first anti-SARS-CoV-2 antibody, as a detection antibody, to form a second antibody-SARS-CoV-2 spike protein-first antibody complex; and
(iii) detecting the presence of the second antibody-SARS-CoV-2 spike protein-first antibody complex, thereby detecting the presence of SARS-CoV-2 in the sample.

In one example, the method of the present invention is performed in an ELISA sandwich assay. In this assay, the capture antibody is coated onto ELISA plates. After blocking, the plates are incubated with the biological sample, washed and then incubated with a detection antibody. For example, the capture antibody is the first anti-SARS-CoV-2 antibody of the present invention, and the detection antibody is the second anti-SARS-CoV-2 antibody of the present invention, and the plate is developed using a ELISA colorimetric TMB reagent.

In another example, the method of the present invention is performed in a flow-through or lateral flow format. In this assay, the detection antibody with a detectable label such as a colorimetric label (e.g. colloidal gold) is immobilized to a membrane such as nitrocellulose (as a strip). A biological sample suspected of containing said SARS-CoV-2 is applied to the membrane to which the detection antibody is present. The biological sample migrates along the membrane through a region containing the detection antibody wherein the detection antibody binds to the spike protein of SARS-CoV-2 if present in the biological sample. The complex of the detection body with its bound antigen then migrates to the test area where a capture antibody is immobilized and also binds the spike protein of SARS-CoV-2, thereby forming a sandwich of the detection antibody, antigen and capture antibody. Concentration/aggregation of detection antibody at the test (capture) area indicates the presence of the spike protein of SARS-CoV-2 in the sample. Such tests can typically be performed with a very small amount of biological sample.

In a related aspect, the present invention also provides a kit for performing the method of the invention, which comprises any of the antibody or its combination thereof as described herein. The kit can further comprise instructions for using the kit to detect the SARS-CoV-2 in a sample.

In some embodiments, the immunoassay is in a sandwich format.

In particular, the kit comprises a pair of anti-SARS-CoV-2 antibodies, which are the first and the second anti-SARS-CoV-2 antibody, to perform a sandwich assay.

In some embodiments, the first anti-SARS-CoV-2 antibody is performed as a capture antibody, paired with the second anti-SARS-CoV-2 antibody as a detection antibody.

In other embodiments, the first anti-SARS-CoV-2 antibody is performed as a detection antibody, paired with the second anti-SARS-CoV-2 antibody as a capture antibody.

As a detection antibody, the antibody can comprise a detectable label such as an enzymatic label, a fluorescent label, a metal label and a radio label.

In some examples, in a lateral flow format, the kit comprises an assay strip (e.g. a nitrocellulose membrane); a detection antibody may be bound to a reaction zone of the strip, and a capture antibody may be bound in a test zone of the strip. The strip also contains a sample pad where a body fluid sample is placed to and then the sample migrates towards an opposite end of the strip by capillary action, through which the sample first engages the detection antibody in the reaction zone where the antigen in the sample bind to the detection antibody forming an antigen-detection antibody complex and then the antigen-detection antibody complex engages the capture antibody in the test (capture) zone. Using colloidal gold as the detectable label of the detection antibody, for example, concentration/aggregation of detection label at the test (capture) area reveals red color, indicating the presence of specific antigen in the sample. Alternatively, the test zone may have a chromogenic substrate and when the antigen-detection antibody complex engages the capture antibody, the chromogenic substrate is converted to a visible colored product.

In some examples, in an ELISA format, the kit comprises a microtiter plate with wells to which a capture antibody has been immobilized; a solution containing a detection antibody; and a color developing reagent.

Particularly, the kit may further comprise additional reagents or buffers, a medical device for collecting a biological sample form a subject, and/or a container for holding and/or storing the sample.

In further embodiments, the present invention provides compositions comprising one or more antibodies as described herein and a pharmaceutically acceptable carrier.

In some embodiments, the composition of the present invention is a pharmaceutical composition for use in treatment of COVID-19.

In some embodiments, the composition of the present invention is a diagnostic composition for use in diagnosis of COVID-19.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said composition may be tablets, pills, powder, lozenges, packets, troches, elixirs, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art, and no extra creative labor is required.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1

### Preparation of Anti-SARS-CoV-2 Antibodies

1. Preparation of Recombinant S Protein

DNA sequence encoding the 268th to the 1255th amino acid residues (SEQ ID NO: 21) of the spike protein of SARS CoV was cloned into an expression vector pET to obtain the plasmid pET-S_{268-1255.} The plasmid pET-S₂₆₈₋₁₂₅₅ was then transformed into Escherichia coli BL21(DE3). The E. coli BL21(DE3) containing plasmid PET-S₂₆₈₋₁₂₅₅ was induced with isopropyl-β-D-thiogalactopyranoside (IPTG) to produce histidine-tagged recombinant S protein. After induction, cells were harvested and lysed by sonication. Following centrifugation, the pellets were resuspended in buffer B (8 M urea, 0.1 M sodium phosphate [pH 8.0], and 10 mM Tris) and stirred at room temperature for 1 hour. After centrifugation, the supernatant was purified via metal chelate affinity chromatography using Ni²⁺-nitrilotriacetic acid (NTA) complexes (Qiagen, Hilden, Germany). Briefly, supernatant was passed through a column of Ni²⁺-NTA agarose that was prewashed with buffer B, buffer C (8 M urea, 0.1 M sodium phosphate, 10 mM Tris [pH 6.3]), and buffer F (6 M guanidine-HCl, 0.2 M acetic acid) and preequilibrated in buffer B. The column was then washed with 10 volumes of buffer B and then with 10 volumes of buffer C, and the protein was eluted with buffer D (8 M urea, 0.1 M sodium phosphate, 10 mM Tris [pH 5.9]) and buffer E (8 M urea, 0.1 M sodium phosphate, 10 mM Tris [pH 4.5]) in fractions of 0.5 ml. Protein-containing fractions were identified by sodium dodecyl sulfate-polyacrylamide gel electrophoresis (SDS-PAGE) and Coomassie blue staining. To renature the protein, a stepwise dialysis was performed at 4° C. against buffer B containing decreasing concentrations of urea (4, 2, 1, 0.5, 0.25, 0.125, and 0.05 M) and against buffer D (20 mM HEPES [pH 8.0], 20% glycerol, 0.1 M KCl, 0.2 mM EDTA, 0.5 mM phenylmethylsulfonyl fluoride, 0.5 mM dithiothreitol [DTT], and 0.2% Nonidet P-40) alone. After dialysis and a short centrifugation at 14,000×g for 5 minutes at 4° C., the supernatant was quickly frozen in liquid nitrogen and stored at −80° C.

2. Generation and Characterization of Monoclonal Antibodies Against SARS-CoV-2 Spike protein

All experiments were performed using BALB/c mice purchased from the National Laboratory Animal Center (Taiwan) and maintained at the Institute of Preventative Medicine's animal housing facility (Taiwan). For the first inoculation, four-week-old BALB/c mice were intraperitoneally (i.p.) immunized with 10 μg of recombinant spike protein in complete Freund's adjuvant. The mice were then administered subsequent immunization boosts against their respective serotypes using 10 μg of recombinant spike protein in incomplete Freund's adjuvant. Final booster containing 10 μg of antigen were administered via i.p. injection. Fusion was performed 5 days after the last injection with the spleen cells of the donor mouse. Briefly, the spleens of immunized mice were removed and splenocytes were fused with NSI/1-Ab4-1 myeloma cells to generate hybridoma cells, which were selected in accordance with the standard procedures outlined by Kohler and Milstein (1975). The fused cells were washed twice with RPMI and then mixed with 1 ml 50% (w/v) PEG 1500 (Roche, Penzberg, Germany) (which was gradually added over a period of 1 minutes under gentle stiffing) in a 15 ml conical tube. The mixture was then diluted twice through the slow (1 minutes) addition of 1 ml RPMI, followed by the slow addition (2 minutes) of 8 ml serum-free RPMI. The mixture was subsequently centrifuged at 400×g for 5 minutes. The fused cell pellet was re-suspended in RPMI supplemented with 20% FBS, HAT medium (Life technologies, Burlington, ONT Canada) and HFCS solution (Roche, Mannhein, Germany), and finally, the resuspension mixture was distributed in 96-well plates (200 μl per well). Hybridoma cell lines that secreted specific antibodies against recombinant spike protein were identified via indirect ELISA (using purified recombinant S protein as the coating antigen). Single clone cells were generated by limiting dilution. Western blotting analysis of lysates from Vero E6 cells infected with SARS-CoV, SARS-CoV-2, HCoV-229E, HCoV-OC43, HCoV NL63, Flu A A/CA/07/2009, Flu B B/T/81863/2014, Parainfluenza-3, Adenovirus-7a, Rhinovirus, RSV was performed to determine (a) the specificity of anti-S mAbs and (b) whether the epitopes recognized by the antibodies were conformational or linear. The mAbs were isotyped using a commercial mouse monoclonal antibody isotyping kit (IsoStrip™, Roche, Mannheim, Germany). The hybridoma cells were i.p. injected into pristane-primed BALB/c mice to induce ascitic fluid production. The mAbs were then purified from ascitic fluids using a protein G-sepharose column (HiTrap protein G, GE Healthcare, Uppsala, Sweden) in accordance with the manufacturer's instructions.

3. Indirect Immunofluorescence Assay (IFA)

The Vero E6 monolayer cells infected with SARS-CoV-2 were washed three times with PBS and then fixed in acetone-Methanol mixture (1:1) for 3 minutes at room temperature. The wells were subsequently blocked using blocking buffer (PBS, 0.05% Tween, 5% skim milk) at 37° C. for 1 hour and washed with wash buffer (PBS, 0.05% Tween). Monoclonal antibodies were then diluted using blocking buffer and incubated at 37° C. for 1 hour. The plates were then washed before adding goat anti-mouse-FITC to undergo incubation again at 37° C. for 1 hour. Finally, each stained monolayer was viewed with an immunofluorescent microscope.

4. Sequence Analysis of Variable Domains of Monoclonal Antibodies

Total RNA was isolated from hybridoma cell lines 8a-1 and 27b-2 using RNeasy mini kit (Qiagen, Valencia, Calif. USA) according to manufacturer's protocol, followed by reverse transcription using oligo dT primer to generate cDNA. The heavy chain and light variable chains of the antibody are amplified using PCR and confirmed the sequence is functional variable domain.

5. Results

Generation and Characterization of Monoclonal Antibodies Against SARS-CoV-2 Spike protein

As shown in Table 1, clones 8a-1 and 27b-2 (mAb8a-1 and mAb27b-2) are both specific to SARS-CoV and SARS-CoV-2, and none of clones reacts with any other respiratory viruses, such as human coronavirus (HCoV), influenza A (Flu A), influenza B (Flu B), Adenovirus, Parainfluenza, Rhinovirus, and Respiratory Syncytial Virus (RSV).

TABLE 1
Characterization of mAb reactions with SARS-CoV-2 Spike protein

| Hybridoma Cell Strain | | | 8a-1 | 27b-2 |
|---|---|---|---|---|
| Isotype | | | IgG1 | IgG2b |
| Type of Epitope | | | linear | linear |
| Reactivity of | Western blot^{a} | SARS-CoV | POS | POS |
| Viruses | | SARS-CoV-2 | POS | POS |
| | | HCoV-229E | NEG | NEG |
| | | HCoV-OC43 | NEG | NEG |
| | | HCoV-NL63 | NEG | NEG |
| | | Flu AA/CA/07/2009 | NEG | NEG |
| | | Flu B B/T/81863/2014 | NEG | NEG |
| | | Parainfluenza-3 | NEG | NEG |
| | | Adenovirus-7a | NEG | NEG |
| | | Rhinovirus | NEG | NEG |
| | | RSV | NEG | NEG |
| | ELISA^{b} | SARS-CoV | POS | POS |
| | | SARS-CoV-2 | POS | POS |
| | IFA^{c} | SARS-CoV | POS | POS |
| | | SARS-CoV-2 | POS | POS |

^{a}The lysates of Vero E6 cells infected with different respiratory viruses were treated with SDS-PAGE sample buffer and blotted with mAb8a-1 and mAb27b-2, respectively.
^{b}Microwell plates were coated with either the recombinant spike protein (expressed in E. coli) or spike protein of SARS-CoV-2 (purchased from Sinovac Biotech Ltd., Beijing, China) and reacted with each mAb.
^{c}Vero E6 cells were infected with SARS-CoV or SARS-CoV-2.

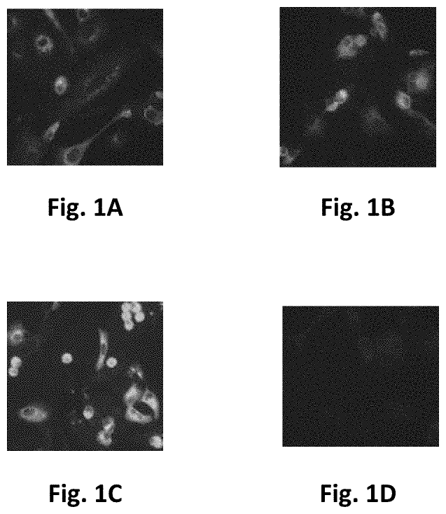

The results of IFA are shown in FIGS. 1A-1D. Both mAb8a-1, mAb27b-2, and serum from a patient infected with SARS-CoV-2 identified Vero E6 cells infected with SARS-CoV-2 (as shown in FIG. 1A, FIG. 1B, and FIG. 1C, respectively). An anti-dengue virus monoclonal antibody, anti-D2 NS1 (FIG. 1D), as a negative control, did not identify Vero E6 cells infected with SARS-CoV-2.

Monoclonal antibody (mAb) 8a-1 has a V_{H} comprising SEQ ID NO: 4, including HC CDR1 of SEQ ID NO: 1, HC CDR2 of SEQ ID NO: 2, and HC CDR3 of SEQ ID NO: 3, and a V_{L} comprising SEQ ID NO: 8, including LC CDR1 of SEQ ID NO: 5, LC CDR2 of SEQ ID NO: 6, and LC CDR3 of SEQ ID NO: 7. Exemplary nucleic acid sequences encoding the V_{H} and the V_{L} are SEQ ID NOs: 17 and 18, respectively.

Monoclonal antibody (mAb) 27b-2 has a V_{H} comprising SEQ ID NO: 12, including HC CDR1 of SEQ ID NO: 9, HC CDR2 of SEQ ID NO: 10, and HC CDR3 of SEQ ID NO: 11, and a V_{L} comprising SEQ ID NO: 16, including LC CDR1 of SEQ ID NO: 13, LC CDR2 of SEQ ID NO: 14, and LC CDR3 of SEQ ID NO: 15. Exemplary nucleic acid sequences encoding the V_{H} and the V_{L} are SEQ ID NOs: 19 and 20, respectively.

### Example 2

### Preparation of SARS-CoV-2 ELISA Test Kit

1. HRP Conjugation

To conjugate mAbs with Horseradish peroxidase (HRP) (Innova Biosciences, Cambridge, UK), 100 μg of HRP and a 20 μl aliquot of modifier reagent were mixed with 200 μl of 1 mg/ml mAb. After incubating the mixture for 3 hours at room temperature (20-25° C.), the reaction was stopped with a 20 μl aliquot of quencher. Following incubation for an additional 30 minutes at room temperature, 260 μl of glycerol was added, and the final solution was stored at −20° C. The final concentration of mAb-HRP was 400 μg/ml.

2. Preparation of SARS-CoV-2 Capture ELISA Test Kit

Monoclonal antibody (mAb) 8a-1 was used as capture antibodies, and pairing to HRP conjugated mAb27b-2 (mAb27b-2-HRP) as detection antibodies. The most appropriate experimental condition such as coating concentration and the dilution of mAb-HRP were determined by checkerboard titration. Ninety-six (96)-well plates (Nunc Immuno Maxisorp, Thermo, Roskilde, Denmark) were coated with 100 μl of 10 μg/ml of capture antibodies (mAb8a-1) and incubated overnight at 4° C. The wells were subsequently blocked using blocking buffer (PBS, 0.05% Tween, 5% skim milk) at 37° C. for 1 hour and washed with wash buffer (PBS, 0.05% Tween) for further use.

3. Specificity of SARS-CoV-2 Capture ELISA Test Kit

Viral infected cell lysate and viral culture supernatants were diluted using blocking buffer and incubated at 37° C. for 1 hour. The plates were then washed before adding 100 μl of 0.8 μg/ml of mAb27b-2-HRP to undergo incubation again at 37° C. for 1 hour. Finally, the microwell plates were washed once again before adding 100 μl of 3,3′,5,5′-Tetramethylbenzidine (TMB) reagent to undergo incubation again at room temperature for 10 minutes. The reaction was stopped using 1 N sulfuric acid, whereupon the absorbance was read at 450 nm (OD₄₅₀) using a microplate reader.

4. Sensitivity of SARS-CoV-2 Capture ELISA Test Kit

The 96-well plates (Nunc Immuno Maxisorp, Thermo, Roskilde, Denmark) were coated with 100 μl of 10 μg/ml of mAb8a-1 and incubated overnight at 4° C. The wells were subsequently blocked using blocking buffer (PBS, 0.05% Tween, 5% skim milk) at 37° C. for 1 hour and washed with wash buffer (PBS, 0.05% Tween). SARS-CoV-2 spike S2 domain protein was then serially diluted using blocking buffer and incubated at 37° C. for 1 hour. The plates were then washed before adding 100 μl of 0.8 μg/ml of mAb27b-2-HRP to undergo incubation again at 37° C. for 1 hour. Finally, the microwell plates were washed once again before adding 100 μl of TMB reagent to undergo incubation again at room temperature for 10 minutes. The reaction was stopped using 1 N sulfuric acid, whereupon the absorbance was read at 450 nm (OD₄₅₀) using a microplate reader.

To determine baseline detection limits of SARS-CoV-2 capture ELISA, bovine serum albumin (BSA) was used to establish baselines for the assay. Samples with OD₄₅₀ values at least double that of BSA were considered positive.

5. Statistical Analysis

Diagnosis accuracy, specificity, sensitivity, and the corresponding 95% confidence intervals (CI95) for each ELISA were performed using GraphPad Prism version 6.0 (GraphPad software, San Diego, Calif., USA), and the significance level was set at a P value of <0.05.

6. Results

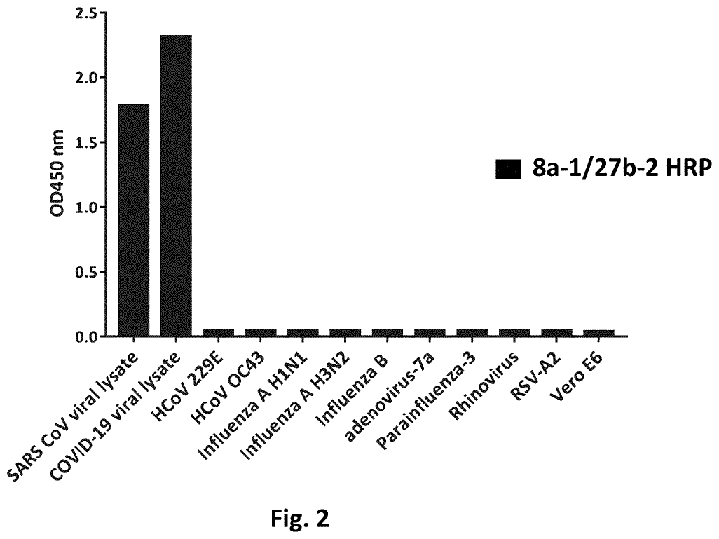

The results of specificity of SARS-CoV-2 capture ELISA test kit are shown in FIG. 2. The results revealed that the combination of the monoclonal antibodies of the present invention provided strong specificity to SARS-CoV and SARS-CoV-2 and presented no signs of reactivity with HCoV, influenza A, influenza B, parainfluenza, adenovirus, Rhinovirus, or RSV.

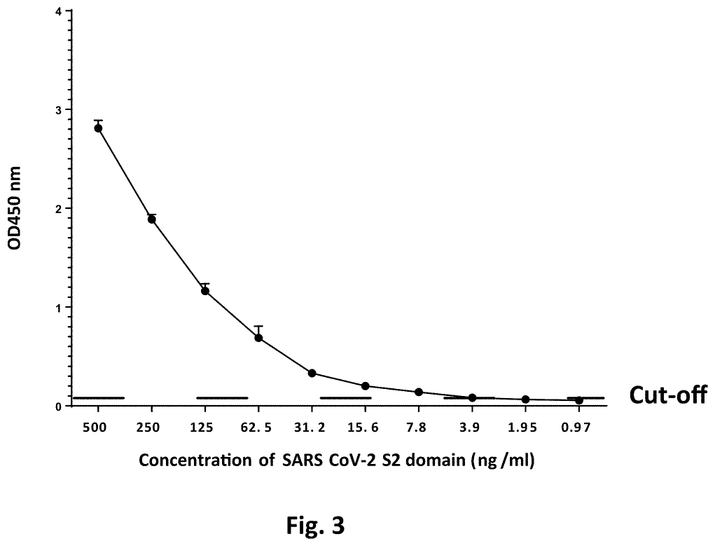

The result of sensitivity of SARS-CoV-2 capture ELISA test kit is shown in FIG. 3. The results revealed that the minimum detection levels of SARS-CoV-2 S2 domain protein capture ELISA was determined to be between 3.9-7.8 ng/ml.

### Example 3

### Preparation of SARS-CoV-2 Test Strips

The two mAbs of the present invention, mAb8a-1 and mAb27b-2, were also used to develop strips for SARS-CoV-2 detection.

1. Preparation of Colloidal Gold Probe

Colloid gold 35±5 nm (TANBead NanoGold-40, Taiwan Advanced Nanotech Inc., Taiwan) was used for conjugation of IgG. The colloid gold solution (1% w/v) was adjusted pH with 0.2 mM K₂CO₃ and mAb8a-1 (in PBS, PH 7.4) was added to pH-adjusted colloid gold solution. The optimized antibody concentration for conjugation was 1μg/strip. The antibody/colloid gold mixture was gently mixed for 90 minutes, blocked by 2% BSA solution for 30 minutes and centrifuged at 7000 rpm for 15 minutes. After centrifugation and wash once by 1% BSA (20 mM Tris/HCl buffer [pH7.2] containing 1% [w/v] BSA), the gold pellets were suspended in 1% BSA. The anti-SARS-CoV-2 mAb8a-1 coated colloidal gold probe was disposed on a pad and dried, then stored at 4° C. overnight.

2. Preparation of Lateral Flow Test Strips

Zero point two (0.2) μg/strip of goat anti mouse IgG (Jackson ImmunoResearch, Pa., USA) used as control lines and 1 μg/strip of mAb27b-2 in PBS (pH 7.4) used as test lines (capture antibodies) were separately applied near to the top end of a cellulose acetate supported strip of nitrocellulose membrane (pore size: 12 μm-diameter) and dried for 1 hour at room temperature. After treatment of components, the lateral flow test device was assembled.

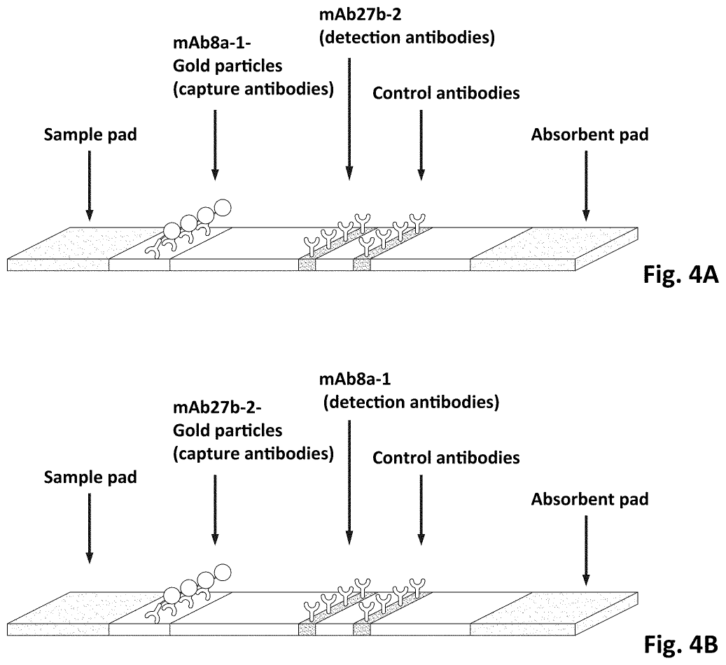

FIG. 4A shows the basic design of the sandwich strip using mAb8a-1 coated colloidal gold probe as the capture antibodies, paired with mAb27b-2 as the detection antibodies. FIG. 4B shows the basic design of the sandwich strip using mAb27b-2 coated colloidal gold probe as the capture antibodies, paired with mAb8a-1 as the detection antibodies.

3. Sensitivity of SARS-CoV-2 Test Strips

The assay was carried out by applying a sample of the appropriate test SARS-CoV-2 spike protein (250 ng/mL, 125 ng/mL, 62.5 ng/mL, and 0 ng/mL) solution 100 μl to the sample pad of the device. The combined solution of test SARS-CoV-2 spike protein and detection reagent rose up the membrane and colloidal gold was deposited at the site of the solid-phase antibody after 20 minutes at room temperature.

4. Specificity of SARS-CoV-2 Test Strips

Viral culture supernatants from other respiratory viruses (HCoV-229E, HCoV-OC43, Flu A/CA/07/2009, Flu B/T/81863/2014, Parainfluenza-3, RSV, Rhinovirus, Adenovirus-7a) were assayed by SARS-CoV-2 Test Strips to evaluate cross-reactivity (specificity). Supernatant from non-infected Vero E6 cells was used as a negative control.

5. Results

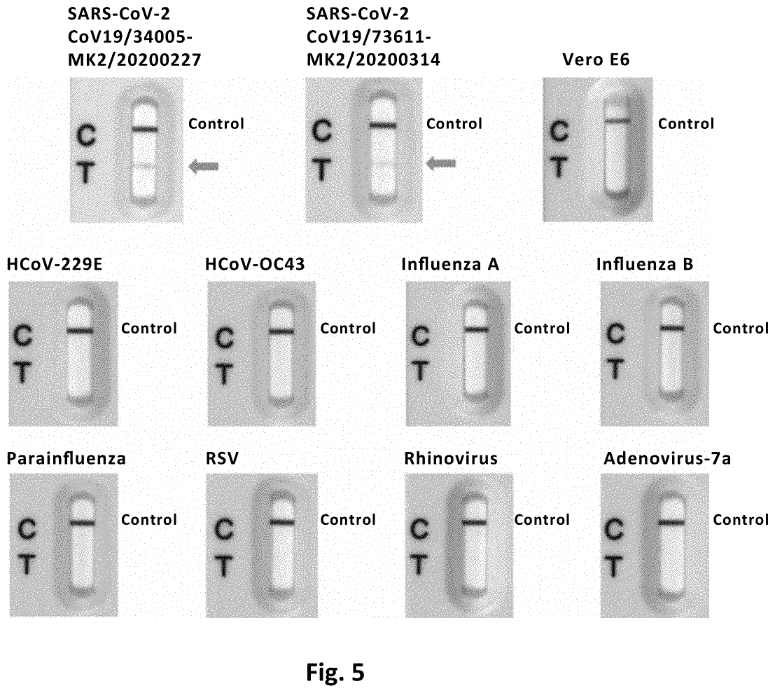

As shown in Table 2 and FIG. 5, mAb27b-2 of the present invention was demonstrated to successfully act as capture antibody, paring with mAb8a-1 of the present invention as detection antibody, in SARS-CoV-2 test strips, exhibiting superior specificity to SARS-CoV-2, without cross-reaction with other respiratory viruses (HCoV, Flu A, Flu B, Parainfluenza, RSV, Rhinovirus, Adenovirus).

TABLE 2
Results of Specificity of SARS-CoV-2 Test Strips

| Viral Sample | Viral Concentration | Result |
|---|---|---|
| SARS-CoV-2/BataCoV/Taiwan/ | 6.9 × 10⁶ pfu/mL | POS |
| 4/2020/20200215 | | |
| SARS-CoV-2/CoV19/34005-MK2/20200227 | 6.3 × 10⁵ pfu/mL | POS |
| HCoV-229E | 3 × 10⁵ TCID₅₀/mL | NEG |
| HCoV-OC43 | 8 × 10⁵ TCID₅₀/mL | NEG |
| Flu A/CA/07/2009 | 5 × 10⁵ pfu/mL | NEG |
| Flu B/T/81863/2014 | 5 × 10⁵ pfu/mL | NEG |
| Parainfluenza -3 | 5.35 × 10⁵ pfu/mL | NEG |
| Adenovirus-1 | 1 × 10⁶ pfu/mL | NEG |
| Adenovirus-7a | 3 × 10⁵ TCID₅₀/mL | NEG |
| Rhinovirus | 6.4 × 10⁵ TCID₅₀/mL | NEG |
| RSV | 3.9 × 10⁵ TCID₅₀/mL | NEG |

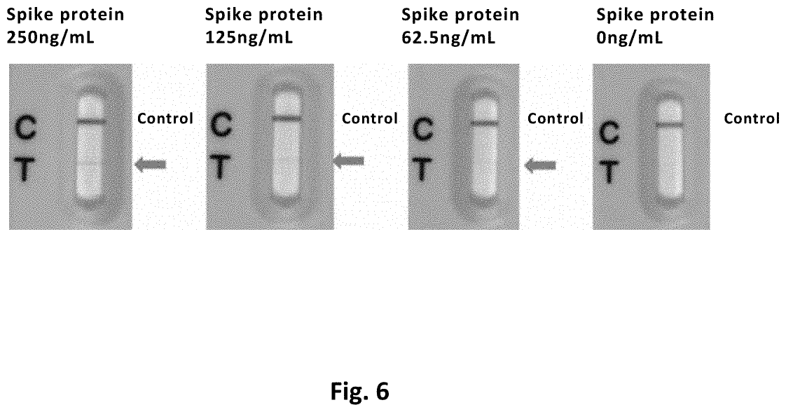

In addition, the detection limits of the monoclonal antibodies of the present invention in strips were determined. As shown in FIG. 6, the results indicate that the SARS-CoV-2 test strip of the present invention achieves superior sensitivity. The strip is able to detect at least 62.5 ng/ml of SARS-CoV-2 spike protein.

What is claimed is:

1. An isolated antibody against Severe Acute Respiratory Syndrome Coronavirus 2 (SARS-CoV-2) or antigen-binding fragment thereof, wherein the isolated antibody is selected from the group consisting of:
(i) a first antibody, comprising
(a) a heavy chain variable region (V_{H}) which comprises a heavy chain complementarity determining region 1 (HC CDR1) of SEQ ID NO: 1, a heavy chain complementarity determining region 2 (HC CDR2) of SEQ ID NO: 2, and a heavy chain complementarity determining region 3 (HC CDR3) of SEQ ID NO: 3; and
(b) a light chain variable region (V_{L}) which comprises a light chain complementarity determining region 1 (LC CDR1) of SEQ ID NO: 5, a light chain complementarity determining region 2 (LC CDR2) of SEQ ID NO: 6, and a light chain complementarity determining region 3 (LC CDR3) of SEQ ID NO: 7; and
(ii) a second antibody, comprising
(a) a V_{H} which comprises an HC CDR1 of SEQ ID NO: 9, an HC CDR2 of SEQ ID NO: 10, and an HC CDR3 of SEQ ID NO: 11; and
(b) a V_{L} which comprises an LC CDR1 of SEQ ID NO: 13, an LC CDR2 of SEQ ID NO: 14, and an LC CDR3 of SEQ ID NO: 15; and
(iii) a combination of (i) and (ii).

2. The isolated antibody or antigen-binding fragment of claim 1, wherein
(i) the first antibody comprises a V_{H} comprising SEQ ID NO: 4 and a V_{L} comprising SEQ ID NO: 8; and/or
(ii) the second antibody comprises a V_{H} comprising SEQ ID NO: 12 and a V_{L} comprising SEQ ID NO: 16.

3. The isolated antibody or antigen-binding fragment of claim 1, wherein the antigen-binding fragment is selected from the group consisting of scFv, (scFv)₂, Fab, Fab′, and F(ab′)₂ of the isolated antibody against SARS-CoV-2.

4. A composition comprising the isolated antibody or antigen-binding fragment thereof of claim 1.

5. The composition of claim 4, which is a pharmaceutical or diagnostic composition for use in treatment or diagnosis of Coronavirus disease 2019 (COVID-19).

6. The composition of claim 4, which comprises a pharmaceutically acceptable carrier.

7. A method for detecting SARS-CoV-2 in a sample suspected of containing said SARS-CoV-2, comprising contacting the sample with an isolated antibody or antigen-binding fragment thereof specific to SARS-CoV-2, and assaying binding of the antibody with the sample, wherein the isolated antibody is selected from the group consisting of:
(i) a first antibody that comprises a V_{H} comprising an HC CDR1 of SEQ ID NO: 1, an HC CDR2 of SEQ ID NO: 2, and an HC CDR3 of SEQ ID NO: 3; and a V_{L} comprising an LC CDR1 of SEQ ID NO: 5, an LC CDR2 of SEQ ID NO: 6, and an LC CDR3 of SEQ ID NO: 7; and
(ii) a second antibody that comprises a V_{H} comprising an HC CDR1 of SEQ ID NO: 9, an HC CDR2 of SEQ ID NO: 10, and an HC CDR3 of SEQ ID NO: 11; and a V_{L} comprising an LC CDR1 of SEQ ID NO: 13, an LC CDR2 of SEQ ID NO: 14, and an LC CDR3 of SEQ ID NO: 15; and
(iii) a combination of (i) and (ii).

8. A kit for detecting the presence of SARS-CoV-2 in a sample, comprising one or more isolated antibodies specific to SARS-CoV-2 or antigen-binding fragment thereof, wherein the isolated antibody is selected from the group consisting of:
(i) a first antibody that comprises a V_{H} comprising an HC CDR1 of SEQ ID NO: 1, an HC CDR2 of SEQ ID NO: 2, and an HC CDR3 of SEQ ID NO: 3; and a V_{L} comprising an LC CDR1 of SEQ ID NO: 5, an LC CDR2 of SEQ ID NO: 6, and an LC CDR3 of SEQ ID NO: 7; and
(ii) a second antibody that comprises a V_{H} comprising an HC CDR1 of SEQ ID NO: 9, an HC CDR2 of SEQ ID NO: 10, and an HC CDR3 of SEQ ID NO: 11; and a V_{L} comprising an LC CDR1 of SEQ ID NO: 13, an LC CDR2 of SEQ ID NO: 14, and an LC CDR3 of SEQ ID NO: 15; and
(iii) a combination of (i) and (ii).

9. The kit of claim 8, wherein the one or more isolated antibodies specific to SARS-CoV-2 or antigen-binding fragment thereof comprises a detectable label.

10. The kit of claim 9, wherein the detectable label is selected from the group consisting of an enzymatic label, a fluorescent label, a metal label, and a radio label.

11. The kit of claim 9, wherein the detectable label is selected from the group consisting of gold nanoparticles, colored latex beads, magnetic particles, carbon nanoparticles, and selenium nanoparticles.

12. The kit of claim 8, wherein the kit is an immunoassay kit.

13. The kit of claim 12, wherein the immunoassay is selected from the group consisting of ELISA (enzyme-linked immunosorbent assay), RIA (radioimmunoassay), FIA (fluorescence immunoassay), LIA (luminescence immunoassay), and ILMA (immunoluminometric assay).

14. The kit of claim 12, wherein the immunoassay is in a lateral flow assay format.

15. The kit of claim 12, wherein the immunoassay is a sandwich assay.

16. A nucleic acid comprising a nucleotide sequence encoding a heavy chain variable region (V_{H}), a light chain variable region (V_{L}) or both, wherein the V_{H} and V_{L} are as set forth in claim 1.

17. An isolated host cell comprising the nucleic acid of claim 16.
